(19) United States
(12) Patent Application Publication
(10) Pub. No.: US 2015/0064794 A1
(43) Pub. Date: Mar. 5, 2015

(54) BIORELEVANT COMPOSITIONS

(71) Applicant: PHARES PHARMACEUTICAL RESEARCH N.V., Curacao (NL)

(72) Inventors: Mathew Louis Steven Leigh, Muttenz (CH); Steve Leigh, Muttenz (CH); Jennifer Dressman, Frankfurt am Main (DE); Bastian Kloefer, Loerrach (DE); Marcel Arndt, Morfelden-Walldorf (DE); Alexander Fuchs, Schlangenbad (DE)

(21) Appl. No.: 14/389,701

(22) PCT Filed: Apr. 2, 2013
(86) PCT No.: PCT/EP2013/056945

§ 371 Date: Sep. 30, 2014

(30) Foreign Application Priority Data
Mar. 30, 2012 (EP) 12162548.7

(51) Int. Cl. G01N 33/94

(52) U.S. Cl. CPC G01N 33/94
USPC 436/34

(57) ABSTRACT

This application relates to a homogeneous biorelevant composition for preparing fasted state biorelevant media having a surface tension between 25 mN/m and 50 mN/m for simulating fasted state gastric and fasted state upper small intestinal fluids of mammalian species, especially humans, dogs, etc. comprising at least one bile salt, eg. sodium taurocholate or sodium taurodeoxycholate; at least one phospholipid, especially 60-99 wt % phosphatidylcholine (PC), enzyme digested diacylphospholipids containing 50-90 wt % of monoacyl-PC; or mixtures thereof; and at least one fatty acid or monovalent salt of the fatty acid, such as sodium oleate. The application also relates to an aqueous biorelevant media composed of surfactants occurring in the gastrointestinal tract of mammals, in particular when prepared from above homogeneous biorelevant composition.

### CROSS-REFERENCE TO RELATED APPLICATIONS

This application is a national phase entry under 35 U.S.C. §371 of PCT/EP2013/056945 filed on Apr. 2, 2013, which claims priority to European Patent Application 12162548.7 filed on Mar. 30, 2012, the entirety of each of which is incorporated by this reference.

### FIELD OF THE INVENTION

This invention relates to biorelevant compositions and method for reconstituting biorelevant media from the compositions. The invention particularly discloses novel compositions and method for preparing reproducible and consistent fasted state biorelevant media defined by selected biorelevant components and physicochemical parameters that simulate fasted state fluids in the stomach and intestine. Fasted state biorelevant media are suitable for solubility and dissolution assessments of poorly water soluble compounds and their dosage forms, with a view to oral administration.

### BACKGROUND TO THE INVENTION

Upper Gastrointestinal Physiology and Importance of Site of Dissolution

Before absorption of a drug from the digestive tract can occur, it must be in solution. For immediate release dosage forms, the first opportunity for release and dissolution is in the stomach. The human stomach functions as a processing organ for food and drugs entering the digestive tract. In the fasted state, humans typically have a low pH in the stomach. Basic drugs which are ionised at low pH, can be readily dissolved under these conditions and so become available for absorption as soon as they enter the small intestine. For basic drugs that are insoluble in the small intestine, dissolution in the stomach before it enters the small intestine can assist the drug being available for absorption from the small intestine. In the small intestine, the dissolution of poorly soluble, weakly acidic drugs will be supported by the higher pH (approx. 5-8) and further enhanced by the natural surfactants, principally bile salts and phospholipids, which can solubilize the poorly soluble drug in colloidal aggregates, including mixed micelles.

Poorly soluble compounds are classified as Class 2 and Class 4 compounds in the Biopharmaceutical Classification System (BCS). For these drugs, solubility and dissolution in the stomach and small intestine are often critical to the bioavailability after oral administration. So it is desirable to test solubility and dissolution of BCS Class 2 and 4 drugs in media simulating these regions to assess the extent their oral absorption will be limited by poor solubility/dissolution.

In the pharmaceutical industry it is common practice to test for solubility in biorelevant media such as Fasted State Simulated Intestinal Fluid (FaSSIF) (Dressman et. al., Dissolution testing as a prognostic tool for oral drug absorption: immediate release dosage forms. Pharm. Res. 15:11-22 (1998)) as part of routine preclinical assessment of new drug candidates.

What are Biorelevant Media?

Biorelevant media aim to reproduce the conditions in the gastrointestinal (GI) tract in vitro, so that the behaviour of drugs and dosage forms in the GI tract can be studied in the laboratory. Typically, they are used for in vitro solubility and dissolution studies but can also be applied to studies of decomposition under GI conditions or for the determination of the permeability characteristics of the drug. Biorelevant media typically comprise solutions of surfactants which are naturally occurring in the GI tract and are adjusted to pH values representative of the local region to be simulated. Typically, biorelevant media are designed to reflect the gastric and intestinal fluids in the fasted or the fed state.

It is recommended that bases of new compounds and generic versions of known drug products are tested not only in biorelevant media representing the small intestine but also in biorelevant media representing the stomach.

It is desired to develop drug products which can be administered either with or without food. Since for poorly soluble drugs inadequate bioavailability is most frequently associated with administration of the drug product in the fasted state it is particularly important to evaluate new drug candidates and formulations under conditions that are biorelevant to the fasted state in the stomach and small intestine.

Unmet Needs

Various studies have been reported using biorelevant media for in vitro assessments of solubility and dissolution of poorly soluble drugs and prediction of in vivo release (see e.g. Shono et al. European Journal of Pharmaceutics and Biopharmaceutics 73 (2009) 107-114; Kleberg review (Journal of Pharmacy and Pharmacology 2010; 62: 1656-1668). However, the studies were in biorelevant media which may be fed state or fasted state media generally composed of bile salt, phospholipid and in fed state media fatty acid and monoglycerides representing lipolysis products from food digestion. The components used are not analytically similar and as such the compositions across the studies cannot be qualitatively compared in terms of physicochemical properties. Further, the components currently used for making biorelevant media have not yet been selected for example optimized with respect to a key performance parameter, the surface tension. Moreover, there is an unmet need for media which have consistent characteristics and can be implemented with assurance of reproducibility in different test site laboratories for assessment and comparison of dissolution and solubility of drugs and formulations in order to provide the closest in vitro-in vivo correlations in the selected medium. It is advantageous that the media be easily and reproducibly prepared in an efficient manner as this will lead to more reliable results and thereby better forecasting of in vivo drug performance.

Another limitation is that to date only biorelevant media to simulate the human gastrointestinal tract have been specified. As formulations must also be developed for animal studies in the pre-clinical phase of drug development, it is especially desirable to have access to biorelevant media which can predict in vivo release and dissolution of the drug candidate from the formulation in animal species such as dog, monkey and mini-pig.

### DEFINITIONS

“Biorelevant compositions” in this specification are “instant” versions, i.e. precursors, of selected key surfactants and optionally co-surfactant mixtures in certain proportions for reconstituting consistent biorelevant media in situ. Exemplary compositions may be homogeneous solid compositions for example powders, granules, pellets, tablets. Exemplary compositions may also be homogeneous liquid compositions for example aqueous concentrates comprising 5% to 60% by weight of the surfactant mixtures. In particular a homogeneous composition is a composition with molecularly dispersed components.

Biorelevant gastric media for simulating physiological fluids under fasted state conditions in the stomach are generally described forthwith as Fasted State Simulated Gastric Fluids (i.e. FaSSGF). Biorelevant intestinal media for simulating physiological fluids under fasted state conditions in the small intestine are generally described forthwith as Fasted State Simulated Intestinal Fluid (i.e. FaSSIF).

Biorelevant gastric media comprising a bile salt and phospholipid (consisting of diacyl phospholipids) at a mole ratio of 4:1 for simulating physiological fluids under fasted state conditions in the stomach are specifically described forthwith as original Fasted State Simulated Gastric Fluid (i.e. FaSSGF-Original).

Biorelevant intestinal media comprising a bile salt and phospholipid (consisting of diacyl phospholipids) at a mole ratio of 4:1 for simulating physiological fluids under fasted state conditions in the small intestine are specifically described forthwith as original Fasted State Simulated Intestinal Fluid (i.e. FaSSIF-Original).

Biorelevant gastric media comprising a bile salt and phospholipid (consisting of diacyl phospholipids) at a mole ratio of 15:1 for simulating physiological fluids under fasted state conditions in the stomach are specifically described forthwith as second version of Fasted State Simulated Gastric Fluid (i.e. FaSSGF-V2).

Biorelevant intestinal media comprising bile salt and phospholipid (consisting of diacyl phospholipids) at a mole ratio of 15:1 for simulating physiological fluids under fasted state conditions in the small intestine are specifically described forthwith as second version of Fasted State Simulated Intestinal Fluid (i.e. FaSSIF-V2).

Biorelevant gastric media comprising at least one bile salt, at least one diacyl or monoacyl phospholipid and at least one fatty acid and/or monoacyl phospholipid, in particular monoacyl PC, for simulating physiological fluids under fasted state conditions in the stomach are specifically described forthwith as third version of Fasted State Simulated Gastric Fluid (i.e. FaSSGF-V3 human).

Biorelevant intestinal media comprising at least one bile salt, at least one diacyl or monoacyl phospholipid and at least one fatty acid and/or monoacyl phospholipid, in particular monoacyl PC, for simulating physiological fluids under fasted state conditions in the small intestine are specifically described forthwith as third version of Fasted State Simulated Intestinal Fluid (i.e. FaSSIF-V3 human).

Biorelevant gastric and intestinal media adapted for dogs are specifically described forthwith as FaSSGF-canine and FaSSIF-canine, respectively.

“biorelevant media” in this specification describe aqueous media simulating fasted state conditions in the stomach and the small intestine.

The singular and the plural terms in this specification are interchangeable.

### PRIOR ART

The prior art in the development of biorelevant intestinal media generally cover solubility and dissolution assessments on poorly soluble compounds and their dosage forms using for example original versions in the prior art known in this description as FaSSIF-Original and FeSSIF-Original for in vitro-in vivo correlation and prediction of in vivo drug release (Dressman et. al., Dissolution testing as a prognostic tool for oral drug absorption: immediate release dosage forms. Pharm. Res. 15:11-22 (1998)).

IVISIV (in vitro-in silico-in vivo) modelling and simulation has recently evolved relying on in vitro solubility and dissolution data input from biorelevant media (Shono et. al., Prediction of food effects on the absorption of celecoxib based on biorelevant dissolution testing coupled with physiologically based pharmacokinetic modelling. Europ. Journal Pharm. and Biopharm. 73 107-114 (2009)). For closer simulation of physiological fluids and to provide better prediction of drug release, FaSSIF-V2 and FeSSIF-V2 have been suggested which adopt different proportions of the key components bile salt and phospholipid (Jantratid et. al., Dissolution Media Simulating Conditions in the Proximal Human Gastrointestinal Tract: An Update. Pharmaceutical Research, Vol. 25, No. 7, (2008)).

Physicochemical factors such as molar concentrations and mole ratios of the surfactants, pH, osmolality, viscosity and surface tension in human intestinal aspirates which can affect the solubility and dissolution characteristics of poorly soluble compound are reviewed in the prior art (Kleberg, Journal of Pharmacy & Pharmacology 62:1656-1668 (2010). However, no particular factor other than food effects and the levels of surfactants were thought to significantly affect the properties of the biorelevant media for in vitro solubility and dissolution testing. FaSSIF-Original and FaSSIF-V2 media and the like across the prior art studies were prepared with various combinations and quality of bile salt, phospholipids and in some cases fatty acid with the over-riding object of improving solubility of poorly soluble drugs and better in vitro-in vivo correlation. Depending on the type of drug, FaSSIF-Original or FaSSIF-V2 may more closely match intestinal fluid, suggesting that the drug predisposes media composition for solubility assessments.

The report in Journal of Pharmaceutical Sciences, vol. 99, no. 8. 17 May 2010, pages 3522-3532 by Kleberg K et al studied the impact of free fatty acid-monoglyceride levels and ratios on nanostructural composition and solubilizing capacity of media simulating fed state intestinal fluids (FeSSIF). Typical fed and fasted state media were prepared using crude bile extract claiming about 60% bile content and employed surface tension (ST), dynamic light scattering and cryogenic transmission electron microscopy to compare the type of nanostructures in the media. The ST of the media varied between 28 mN/m and 41 mN/m independent of the concentration of bile salt or the ratio between fatty acid and monoglyceride, depending mainly on the total surfactant concentrations. Further, it was shown that the type of nanostructures was attributed to fatty acids and monoglycerides in FeSSIF and responsible for solubilising unionised poorly soluble drugs. In comparison, ST of prior art FaSSIF medium comprising crude bile salt and without fatty acid or monoglyceride was shown to be about 40 mN/m.

Luner P E et al in Journal of Pharmaceutical Sciences, vol. 90, no. 3, 1 Jan. 2001 (2001-01-01) pages 348-359, essentially assessed the effects of lipids on the wetting behaviour of bile salt and phospholipid solutions. Fatty acids and monoglycerides that are representative of fasted and fed states intestinal fluids under physiological conditions were added to the solutions. Wetting behaviour of the solutions attributed to surface properties on a solid polymethyl methacrylate (PMMA) model substrate were examined to draw correlations between surface tension, contact angle and adhesion tension. Reportedly, micellar systems which depend on both the type and concentration of lipid present in the bile salt solution influenced wetting behaviour. The phospholipids in the simulated media consist of 90-96% pure phosphatidylcholine (PC) and >99% pure lysolecithin (not partially enzyme digested diacyl phospholipids comprising monoacyl PC). The bile component consists of >97% pure taurodeoxycholate and >99% taurocholic acid as the sodium salts. Fatty acids consist of a maximum of 12 carbon chain fatty acids and below, consisting of dodecanoic (C12), heptanoic (C7) and decanoic (C10) acid. The surface tension values were obtained from lipid solutions comprising pure bile salts, pure PC and pure lysolecthin, and fatty acids with 12 carbon chain length and below. Lower surface tension in the fed state media was attributed to monoglycerides. However, the study did not include specific combinations of analytically defined bile salts; partially enzyme digested diacyl phospholipids comprising up to 90% by weight monoacyl PC (i.e. lyso PC); fatty acids with at least 14 carbon chain length (without monoglycerides) in fasted state media. The general view was that although surfactants affect surface tension, to adjust surface tension and solubilizing capacity of dissolution media, there is as yet no consensus as to which surfactant(s) or what concentration(s) should be used to emulate in vivo conditions.

Sunesen V H et al in European Journal of Pharmaceutical Sciences, vol. 24, no. 4, 1 Mar. 2005, (pages 305-313) discloses a study focusing on examining in vitro-in vivo correlations (IVIVC) of a drug using a flow-through dissolution method with typical biorelevant dissolution media prepared from crude components for simulating both fed and fasted states intestinal fluids. The object was to assess the hydrodynamics of the medium which can affect the dissolution of a poorly soluble drug danazol in FaSSIF. The conclusion was that in FeSSIF, IVIVC for the drug could only be obtained by including monoglycerides and fatty acids together in the medium. In the fasted state, the most relevant correlation was achieved in a medium without fatty acids and containing 6.3 mM bile salts from crude bile extracts containing about 53% bile salt and 1.25 mM phospholipids containing 43% PC from crude lecithin. Overall, surface tension of FeSSIF and FaSSIF media prepared from crude components ranged between 25 mN/m and 36 mN/m taking into account the monoglycerides content in FeSSIF and/or impurities in the crude materials for making FaSSIF. No particular range of surface tension was proposed for preparing FaSSIF medium consistently targeting surface tension.

Kalantzi L et al in Pharmaceutical Research, vol. 23, no. 6, 25 May 2006, pages 1373-1381 assessed the relative usefulness of canine intestinal contents and simulated media such as, for example FaSSIF-Original in the prediction of solubility of two basic drugs in fasted and fed human intestinal aspirates. Surface tension values of the simulated fasted state mediumFaSSIF was reported as 49.8 mN/m; canine intestinal fluid ranged from 28.3 mN/m to 36.5 mN/m depending on the interval for taking samples, whilst the surface tension of fasted human intestinal fluid is 33.6 mN/m. FaSSIF in the example shown was prepared using crude bile extracts and phospholipids consisting of 97% by weight diacyl PC and 3% by weight lyso PC.

WO 2007/054342 discloses solid dissolution compositions and method of preparing human biorelevant media comprising both FaSSIF and FeSSIF. The solid compositions describe bile salt and phospholipid complexes consisting of bile salt and phospholipid in the molar ratio of 1:1 to 20:1. The phospholipid may be from a wide selection of phospholipids which may be lecithin, enzyme hydrolysed lecithin, diacyl phospholipids, monoacyl phospholipids.

WO 2008/040799 describes instant forms of biorelevant media comprising bile salt and phospholipids in the ratio 1:1 and 10:1 and optionally breakdown products of triglyceride digestion such as a monoglyceride and a fatty acid in a ratio of 1:10 to 6:1 in relation to the bile salt for preparing only Fed State Simulated Intestinal Fluid (FeSSIF).

The state of the art fails to teach separate biorelevant media for animals typically used in pre-clinical evaluations of pharmaceutical products from those media to be used to evaluate different formulations for human medicine and for selection of the optimal formulation to be used in clinical trials, to aid in de-risking bioequivalence studies prior to or after marketing authorization has been obtained and thus to streamline pharmaceutical development of drug products.

### Advantages of the Invention

It is an advantage of the present invention to provide novel compositions which comprise for example a selection of biorelevant components which are analytically defined and found in the fasted state gastrointestinal region. It is another advantage to optimize biorelevant media for the purpose of simulating fasted state conditions in humans as well as animal species. It is a further advantage of the invention to provide a method of selecting analytically defined components for preparing reproducible biorelevant media for the purpose of better simulating fasted state conditions in the stomach and upper intestine of mammals. A further advantage is to provide biorelevant media useable for testing in vitro solubility, permeability, supersaturation, precipitation, release and dissolution of poorly water soluble compounds and their dosage forms.

Moreover, it is an advantage to improve in vitro test conditions in biorelevant media. In particular, it is an advantage to reduce or avoid variations associated with surfactants from multiple sourcing, minimize the number of studies required to optimize a drug formulation and reduce the risks in relying heavily on in vivo bioequivalence studies.

### SUMMARY

Above advantages are achieved by composing biorelevant media for in vitro studies based on analytically defined components in order to result in a reproducible medium characterized not only by its components but also its consistent physicochemical properties, in particular e.g. a consistent surface tension.

This invention describes novel compositions which may be solid or aqueous concentrates for preparing fasted state biorelevant media. The fasted state medium contain for the first time, selected combinations of bile salt and surfactant simulating fasted state conditions in the stomach and the small intestine. Fasted state biorelevant media target surface tension within a range suitable for dissolution and solubility testing thereby providing a uniform standard when making and comparing drug solubility and dissolution in simulated fasted state medium. Assessing solubility and dissolution of new drug candidates and generic formulations using a uniform and standardized for example, optimized biorelevant medium as disclosed in the invention in comparison to non-optimized medium can help better identify inadequate solubility and/or dissolution of the drug compound and the drug formulation in bioequivalence tests with respect to making the drug available for absorption. Evaluation of potential formulations of new drug candidates in optimized media which have reproducible physical and chemical characteristics across testing programs is a more efficient and reliable way to identify the optimal formulation for oral administration. Accordingly the invention is particularly concerned with solid or aqueous concentrates for preparing fasted state biorelevant media.

The biorelevant media advantageously may be characterised by physicochemical properties, in particular a target range for surface tension for in vitro studies.

Having matched a drug to the fasted state media for solubility testing, the above-mentioned prior art does not go on and specify particular combinations and specific components to make the media more reproducible and reliable for solubility evaluations of drugs. What is not disclosed is a method to exploit analytically specified component(s) and define composition(s) which consistently and reproducibly simulate fasted state gastric and intestinal fluids. The prior art is silent with regard to a method which provides the facility to optimize the composition in relation to the surface tension parameter. Surprisingly it was found that an optimization according to the present invention results in a better control of the reproducibility of fasted state biorelevant media. Components which target the surface tension parameter and may affect the aggregation state, for example organisation of the mixed micelles in simulated media have not been considered to play significant roles in prior art fasted state biorelevant media for solubility and dissolution testing (Fotaki and Vertzoni. The Open Drug Delivery Journal, 2010, 4, 2-13).

Particularly, it was found that by targeting surface tension control of reconstituted biorelevant media simulating fasted state conditions, solubility properties of poorly soluble compounds can be predetermined and optimized. Surprisingly it was found that by controlling the surface tension within the range of 25 mN/m to 50 mN/m the solubility values are optimized and consistently on an advantageous level. Furthermore it was found that the surface tension control of a reconstituted biorelevant media simulating fasted state conditions can be predefined by predefining a precursor composition of the media. It was found that resulting surface tension values depend strongly on the precursor composition. Due to the many components and parameters of a biorelevant media, a further difficulty was to find the key components and parameters which can be used to effectively manipulate surface tension in order to actually target a desired range. None of the prior art documents does teach manipulation of the surface tension as such. Furthermore, none of the prior art documents does identify ways of manipulating the surface tension.

As far as it is known, there is no explicit disclosure in the prior art anticipating biorelevant media for humans and dogs (including other mammalian species) composed of selected components and unique combinations consistently targeting surface tension. Above all, the prior art references separately or combined do not point to the solid or concentrated aqueous compositions of present invention. Further, the benefits of biorelevant media which are optimized and characterised by physicochemical properties in particular, but not limited to surface tension within the inventive range, defined by judicious selections of bile salt and combination of surfactants simulating fasted state conditions in the stomach and the small intestine of human and other mammalian species have not been disclosed in prior art.

Biorelevant compositions defined by selecting particular combinations of analytically specified surfactants are disclosed. The compositions according to present invention may be solid or aqueous concentrates particularly useful for reconstituting fasted state biorelevant media. The reconstructed biorelevant media are composed of analytically defined components and more consistently reproducible in terms of composition and physicochemical properties. Advantageously, fasted state media are confined between limits by selecting the total amount of analytically defined surfactants (mmol), surface tension (mN/m), amount of each surfactant and mol ratio, pH, osmolality (Osmol/kg), buffer capacity and ionic strength. The media are optimized at least in terms of media composition and surface tension within the range defined by the selection of components to simulate fasted state fluids, in particular fasted state human and canine fluids, in the stomach and small intestines for drug solubility and dissolution testing and for comparison of bioequivalence between formulations of the same drug.

Biorelevant media according to present invention (in particular FaSSGF-V3 human and FaSSIF-V3 human), are distinguished from prior art media simulating fasted state conditions, generally known as FaSSGF-Original, FaSSGF-V2 and FaSSIF-Original, FaSSIF-V2, which are composed essentially of bile salts and diacyl phospholipid components. In particular the biorelevant media according to present invention are distinct by their combination of surfactants, including fatty acids and/or monoacyl PC provided in the form of partially enzyme digested diacyl phospholipids, and the physicochemical property of surface tension in the range of 25 mN/m to 50 mN/m, 35 mN/m to 45 mN/m, 28 mN/m to 45 mN/m, or 30 mN/m to 42 mN/m.

It was found that prior art FaSSGF-Original and FaSSIF-Original and FaSSIF-V2 are biorelevant media which are not optimized in that the disclosed compositions and key surfactant components do not contain fatty acids and/or monoacyl PC provided in the form of partially enzyme digested diacyl phospholipids and to a certain degree are variable in quality and effective composition. It was also found that the surface tension parameter of prior art fasted state media can vary considerably for example, outside the range between 25 mN/m and 50 mN/m, particularly between 28 mN/m and 45 mN/m or 30 mN/m and 42 mN/m. Most surprisingly, it was found that control of composition as mentioned above and adjustment of the surface tension parameter as mentioned above result in improved drug solubility and dissolution test conditions.

Control and manipulation of surface tension in the fasted state biorelevant media is achieved by selecting the appropriate amounts of fatty acid(s) and/or partially enzyme digested diacyl phospholipids comprising between 50% and 90% of monoacyl phospholipids in particular monoacyl PC as disclosed in the invention.

Thus biorelevant media according to present invention are optimized and standardized in terms of their composition and physicochemical properties for example, pH, buffer capacity, osmolality, and in particular surface tension within the range defined by selections of the bile salt and surfactants simulating fasted state conditions in the stomach and the small intestine. The targeted value/s of the physiochemical properties is/are specific for the combination of the surfactants in fasted state biorelevant media (e.g. human FaSSGF, such as FaSSGF-V3 human, and human FaSSIF, such as FaSSIF-V3 human), which should be reproduced consistently each time the media is prepared.

### DETAILED DESCRIPTION OF THE EXEMPLARY EMBODIMENTS

In a first exemplary embodiment the following aspects and sub-aspects are disclosed:

In a first aspect a standardised aqueous biorelevant media for simulating fasted state stomach and fasted state upper small intestinal fluids of mammalian species, composed of surfactants occurring in the gastrointestinal tract of mammals comprising

(a) at least 40 mole % to 95 mole % of one bile salt, and

(b) the rest mole % being a combination of at least two surfactants, namely
a diacyl phospholipid and a fatty acid including monovalent salts of the fatty acid, or
a monoacyl phospholipid and a fatty acid including monovalent salts of the fatty acid, or
a diacyl phospholipid and a monoacyl phospholipid and a fatty acid including monovalent salts of the fatty acid, or
a diacyl phospholipid and a monoacyl phospholipid,

further characterised by a surface tension between 25 mN/m and 50 mN/m.

In a second aspect the biorelevant media according to aspect 1 wherein the surface tension is between 35 mN/m and 45 mN/m, between 28 mN/m and 45 mN/m or between 30 mN/m and 42 mN/m.

In a third aspect the biorelevant media according to any of the preceding aspects wherein the mole ratio of the two named surfactants in the mixture is 1:20 to 20:1.

In a fourth aspect the standardised biorelevant media according to any of the preceding aspects further comprising between 0.001 mol % and 10 mole % co-surfactants naturally occurring in the gastrointestinal tract of mammals selected from the group consisting of cholesterol or their esters, monoglycerides, diglycerides, triglycerides, decomposition products of phospholipids other than fatty acids, and mixtures thereof.

In a fifth aspect the biorelevant media according to any of the preceding aspects, wherein the mole ratio of the bile salts to the sum of the surfactants comprised in said combination of at least two surfactants and co-surfactants if present is 2:3 to 19:1, 1:1 to 15:1, 2:1 to 6:1 or 3:1 and 5:1.

In a sixth aspect the biorelevant media according to any of the preceding aspects wherein the mole ratio of said at least one monoacyl phospholipid and diacyl phospholipid to said fatty acids, including monovalent salts of fatty acids, in the mixture is 1:20 to 20:1.

In a seventh aspect the biorelevant media according to any of the preceding aspects wherein the mole ratio of diacyl phospholipids to fatty acids, including monovalent salts of fatty acids in the mixture is 1:20 to 20:1.

In an eighth aspect the biorelevant media according to any of the preceding aspects, further comprising components selected from water, buffer, osmotic components, stabilizers, antioxidants, pH adjusters, antimicrobials, enzymes for example pepsin, pancreatic enzymes.

In a ninth aspect a homogeneous biorelevant composition for preparing fasted state biorelevant media characterised by a surface tension between 25 mN/m and 50 mN/m comprising the following surfactants:

(a) at least 40 mole-% to 95 mole-% of one bile salt, and

(b) the rest mole-% being a combination of at least two surfactants, namely
a diacyl phospholipid and a fatty acid including monovalent salts of the fatty acid, or
a monoacyl phospholipid and a fatty acid including monovalent salts of the fatty acid, or
a diacyl phospholipid and a monoacyl phospholipid and a fatty acid including monovalent salts of the fatty acid, or
a diacyl phospholipid and a monoacyl phospholipid.

In a tenth aspect the homogeneous composition according to aspect 9 in the form of powders wherein the mean particle size is between 10 μm and 1000 μm; bulk density is between 0.3 g/cm³ and 0.7 g/cm³ and moisture content is below 5% by weight; or granules, pellets, tablets, or capsules wherein the mean particle diameter of the granules or pellets is 200 μm to 2000 μm.

In an eleventh aspect the homogeneous composition according to any one of aspects 9 and 10 in the form of a liquid composition comprising between 10% and 60%; or 20% to 60% by weight of surfactants dispersed in aqueous medium comprising further antioxidants and antimicrobials.

In a twelfth aspect a method of preparing a homogeneous solid biorelevant composition according to any one of aspects 9 to 11 comprising, dissolving the surfactants in a volatile solvent, water or mixtures thereof and eliminating the solvent, thereby providing a solid composition wherein the moisture content is below 5% by weight.

In a thirteenth aspect a method of preparing a homogeneous liquid biorelevant composition according to any one of aspects 9 and 11 wherein between 10% and 60% by weight of the surfactants are homogeneously dissolved or dispersed in aqueous medium comprising further components selected from buffer, osmotic components, stabilizers, antioxidants, pH adjusters, and antimicrobials at a temperature between 15° C. and 60° C. without a drying step to remove the water.

In a fourteenth aspect a method for preparing FaSSIF human media comprising 2 to 20 mmol, or 2 to 6 mmol and FaSSIF-canine media comprising 2 to 20 mmol or 10 to 15 mmol of the biorelevant compositions of any one of aspects 9 to 13 comprising a step for adding aqueous medium to the homogeneous solid or diluting the liquid compositions with the aqueous medium wherein the aqueous medium comprises buffers and osmotic regulators.

In a fifteenth aspect a method for preparing FaSSGF human media comprising between 0.01 mmol and 5 mmol, or 0.01 mmol and 1 mmol and FaSSGF-canine media comprising between 0.1 mmol and 5 mmol, or 0.1 and 2 mmol of the biorelevant composition of any one of aspects 9 to 13 comprising a step for adding aqueous medium to the homogeneous solid or diluting the liquid compositions with the aqueous medium wherein the aqueous medium comprises buffers and osmotic regulators.

In a sixteenth aspect a method for preparing biorelevant media according to any one of aspects 1 to 8 comprising individually weighing and dissolving the surfactants and optionally co-surfactants separately, together or sequentially in aqueous medium comprising components selected from water, buffer, osmotic components, stabilizers, antioxidants, pH adjusters, antimicrobials and enzymes for example pepsin, pancreatic enzymes.

In a second exemplary embodiment the following aspects and sub-aspects are disclosed:

In a first aspect a homogeneous biorelevant composition for preparing fasted state biorelevant media having a surface tension between 25 mN/m and 50 mN/m for simulating fasted state gastric and fasted state upper small intestinal fluids of mammalian species, comprising the following surfactants:

at least one bile salt, or two bile salts;

(i) at least one phospholipid selected from the group of phospholipids comprising
between 60% and 99% by weight phosphatidylcholine (PC),
partially enzyme digested diacyl phospholipids containing between 50% and 90% by weight monoacyl PC, that may be obtained by back-blending with PC, and
mixtures of PC and partially enzyme digested diacyl phospholipids wherein the level of monoacyl PC is between 5% and 80% by weight; and

(ii) at least one fatty acid or monovalent salt of the fatty acid.

In a second aspect the homogeneous composition according to aspect 1, characterised in that 40 mole-% to 95 mole-% of said surfactants consist of the at least one bile salt (i) and that the rest mole-% (i.e. 60 mol-% to 5 mol-%) of said surfactants consists of the at least one phospholipid (ii) and the at least one fatty acid or monovalent salt of the fatty acid (iii).

In a third aspect the homogeneous composition according to aspect 2, characterised in that the rest mole-% of the surfactants includes further cholesterol.

In a fourth aspect the homogeneous composition according to any one of aspects 1-3, characterised in that the at least one bile salt or the two bile salts are selected from the group consisting of sodium cholate, sodium taurocholate, sodium glycocholate, sodium deoxycholate, sodium taurodeoxycholate, sodium glycodeoxycholate, sodium ursodeoxycholate, sodium chenodeoxycholate, sodium taurochenodeoxycholate, sodium glyco chenodeoxycholate, sodium cholylsarcosinate, sodium N-methyl taurocholate and their free acids.

In a fifth aspect the homogeneous composition according to any one of aspects 1-4, characterised in that the at least one fatty acid is at least one of 14 carbon to 22 carbon fatty acid.

In a sixth aspect the homogeneous composition according to any one of aspects 1-5 devoid of monoglyceride.

In an seventh aspect the homogeneous composition according to any one of aspects 1-6 in the form of a solid, for example such as
powder, wherein the mean particle size is between 10 μm and 1000 μm; bulk density is between 0.3 g/cm³ and 0.7 g/cm³ and moisture content is below 5% by weight,
granules or pellets, wherein the mean particle diameter of the granules or pellets is 200 μm to 2000 μm,
tablets, or
capsules.

In a eighth aspect the homogeneous composition according to any one of aspects 1-6 in the form of a liquid composition, for example an aqueous concentrate, comprising between 10% and 60%, 20% and 60%, 20% to 50%, or 30% to 40%, by weight of surfactants dispersed in a liquid medium comprising further antioxidants and antimicrobials.

In a ninth aspect an aqueous biorelevant media for simulating fasted state gastric and fasted state upper small intestinal fluids of mammalian species, composed of surfactants occurring in the gastrointestinal tract of mammals, comprising

(iii) at least one bile salt, or two bile salts;

(iv) at least one phospholipid selected from the group of phospholipids comprising
between 60% and 99% by weight phosphatidylcholine (PC),
partially enzyme digested diacyl phospholipids containing between 50% and 90% by weight monoacyl PC, that may be obtained by back-blending with PC, and
mixtures of PC and partially enzyme digested diacyl phospholipids wherein the level of monoacyl PC is between 5% and 80% by weight; and

(v) at least one fatty acid or monovalent salt of the fatty acid, and

having a surface tension between 25 mN/m and 50 mN/m.

This aqueous biorelevant media advantageously is prepared from above homogeneous biorelevant composition according the first aspect.

In a tenth aspect the aqueous biorelevant media according to aspect 9 wherein the surface tension is between 35 mN/m and 45 mN/m, between 28 mN/m and 45 mN/m or between 30 mN/m and 42 mN/m.

In an eleventh aspect the aqueous biorelevant media according to any one of aspects 9 or 10 characterised in that 40 mole-% to 95 mole-% of said surfactants consist of the at least one bile salt (i), and that the rest mole-% (i.e. 60 mol-% to 5 mol-%) of said surfactants consists of the at least one phospholipid (ii) and the at least one fatty acid or monovalent salt of the fatty acid (iii).

In a twelfth aspect the aqueous biorelevant media according to any one of aspects 9-11, characterised in that the rest mole-% of the surfactants includes further cholesterol.

In a thirteenth aspect the aqueous biorelevant media according to any one of aspects 9-12, characterised in that the at least one bile salt or the two bile salts are selected from the group consisting of sodium cholate, sodium taurocholate, sodium glycocholate, sodium deoxycholate, sodium taurodeoxycholate, sodium glycodeoxycholate, sodium ursodeoxycholate, sodium chenodeoxycholate, sodium taurochenodeoxycholate, sodium glyco chenodeoxycholate, sodium cholylsarcosinate, sodium N-methyl taurocholate and their free acids.

In a fourteenth aspect the aqueous biorelevant media according to any one of aspects 9-13, characterised in that the at least one fatty acid is at least one of 14 carbon to 22 carbon fatty acid.

In a fifteenth aspect the aqueous biorelevant media according to any one of aspects 9-14 devoid of monoglyceride.

In a sixteenth aspect the aqueous biorelevant media according to any one of aspects 9-15 wherein the total amount of surfactants for simulating human FaSSGF is between 0.01 mmol and 5 mmol, or between 0.01 and 1 mmol.

In a seventeenth aspect the aqueous biorelevant media according to any one of aspects 9-15 wherein the total amount of surfactants for simulating human FaSSIF is between 2 and 20 mmol, or between 2 and 6 mmol.

In a eighteenth aspect the aqueous biorelevant media according to any one of aspects 9-15 wherein the total amount of surfactants for simulating canine FaSSGF is between 0.1 and 5 mmol, between 0.1 and 2 mmol, or between 0.01 mmol and 5 mmol.

In a nineteenth aspect the aqueous biorelevant media according to any one of aspects 9-15 wherein the total amount of surfactants for simulating canine FaSSIF is between 2 and 20 mmol, between 5 and 20 mmol, or between 10.0 mmol and 15.0 mmol.

In a twentieth aspect the biorelevant media according to any one of aspects 16-19, comprising at least 60 mole-% or at least 70 mole-% of at least one bile salt.

In a twenty-first aspect the aqueous biorelevant media according to any one of aspects 9-20, wherein the mole ratio of the mole sum of monoacyl PC and diacyl PC to the fatty acids, including monovalent salts of the fatty acids, is 1:20 to 20:1.

In a twenty-second aspect the aqueous biorelevant media according to any one of aspects 9-21, wherein the mole ratio of diacyl PC to monoacyl PC is 1:20 to 20:1.

In a twenty-third aspect the aqueous biorelevant media according to any one of aspects 9-22, wherein the mole ratio of diacyl PC to the fatty acids, including monovalent salts of fatty acids, is 1:20 to 20:1.

In a twenty-fourth aspect the aqueous biorelevant media according to any one of aspects 9-23, comprising components selected from water, buffer, osmotic components, stabilizers, antioxidants, pH adjusters, antimicrobials, enzymes for example pepsin, pancreatic enzymes.

In a twenty-fifth aspect a method of reconstituting a biorelevant media by adding defined amounts of the biorelevant composition according to any one of aspects 1-8 to water or aqueous media.

In a twenty-sixth aspect a method of preparing a solid biorelevant composition according to aspect 7, comprising, dissolving the surfactants in a solvent, water or mixtures thereof and eliminating the solvent, thereby providing a solid composition wherein the moisture content is below 5% by weight.

In a twenty-seventh aspect a method of preparing an aqueous concentrate according to aspect 8, wherein between 10% and 60% by weight of the surfactants are homogeneously dissolved or dispersed in aqueous medium comprising further components selected from buffer, osmotic components, stabilizers, antioxidants, pH adjusters, and antimicrobials at a temperature between 15° C. and 60° C. without a drying step to remove the water.

In a twenty-eighth aspect a method for preparing an aqueous biorelevant media simulating fasted state media according to any one of aspects 9-24, comprising a step of adding aqueous medium to the said solid or diluting the said liquid biorelevant compositions with the aqueous medium wherein the aqueous medium comprises buffers and osmotic regulators.

In a twenty-ninth aspect a method for preparing an aqueous biorelevant media according to any one of aspects 9-24, comprising individually weighing and dissolving the surfactants and optional further co-surfactants separately, together or sequentially in aqueous medium comprising components selected from water, buffer, osmotic components, stabilizers, antioxidants, pH adjusters, and antimicrobials and enzymes for example pepsin, pancreatic enzymes.

In a thirtieth aspect a use of the aqueous fasted state biorelevant media according to any one of aspects 9-24, comprising specified proportions of analytically defined surfactants for solubility testing, dissolution testing, bioequivalence assessments, drug release assessments, IVIVC, in silico modelling and simulation, drug supersaturation, drug precipitation, drug stability, performance of enhanced formulations and drug permeability studies.

### DETAILED DESCRIPTION

Solid Compositions and Aqueous Concentrates

This invention describes novel biorelevant compositions which may be solid or liquid.

In particular the solid compositions and liquid compositions, for example aqueous concentrates, are used for reconstituting reproducibly fasted state biorelevant media.

The compositions for example comprise or consist of a selection of analytically defined surfactants occurring in the gastrointestinal tract of mammals chosen from,
(i) at least one or two bile salts that may be selected from the group consisting of sodium cholate, sodium taurocholate, sodium glycocholate, sodium deoxycholate, sodium taurodeoxycholate, sodium glycodeoxycholate, sodium ursodeoxycholate, sodium chenodeoxycholate, sodium taurochenodeoxycholate, sodium glyco chenodeoxycholate, sodium cholylsarcosinate, sodium N-methyl taurocholate and their free acids.
(ii) at least one phospholipid selected from substantially pure phospholipids comprising
   between 60% and 99% by weight phosphatidylcholine (PC),
   partially enzyme digested diacyl phospholipids containing between 50% and 90% by weight monoacyl PC obtained by back-blending with PC
   mixtures of PC (i) and partially enzyme digested diacyl phospholipids (ii) wherein the level of monoacyl PC is between 5% and 80% by weight.
   (iii) at least one fatty acid or monovalent salt of fatty acid, such as at least one 14 carbon to 22 carbon fatty acid or monovalent salt of 14 carbon to 22 carbon fatty acid. The molar concentration of each surfactant as a percentage of the total surfactants in the solid and aqueous concentrates in particular for making fasted state biorelevant media are;
   (a) at least one bile salt: between 40 and 95 mole-% (at least 40 mole-% and between 40 mole-% and 50 mole-%), (at least 50 mole-% and between 50 mole-% and 60 mole-%), (at least 60 mole-% and between 60 mole-% and 70 mole-%), (at least 70 mole-% and between 70 mole-% and 80-mole %), (at least 80-mole % and between 80 mole-% and 90 mole-%) or (at least 90 mole-% and between 90 mole-% and 95 mole-%).
   (b) at least one phospholipid: between 0.1 mole-% and 40 mole-% (or between 0.5 mole-% and 30 mole-%, between 1 mole-% and 20 mole-%, or between 2 mole-% and 20 mole-%; or between 1 mole-% and 15 mole-%.
   Phospholipids comprise:
   (1) substantially pure diacyl phospholipids comprising between 60% and 99% by weight PC,
   or
   (ii) partially enzyme digested diacyl phospholipids containing between 50% and 90% by weight monoacyl PC obtained by back-blending with PC
   Or
   (iii) mixtures of PC (i) and partially enzyme digested diacyl phospholipids
   (ii) wherein the level of monoacyl PC is between 5% and 80% by weight.
   (c) at least one (such as 14 carbon to 22 carbon) fatty acid or monovalent salt of said fatty acid between 0.1 mole-% and 40 mole-% (or between 0.5 mole-% and 30 mole-%, between 1 mole-% and 20 mole-%, or between 2 mole-% and 20 mole-%; or between 1 mole-% and 15 mole-%,); optionally
   (d) cholesterol: between 0 mole-% and 10 mole-%, (or between 0.001 mole-% and 10 mole-%, or between 0.01 mole-% and 7.5 mole-%, or between 0.01 mole-% and 5 mole-%, or between 0.01 mole-% and 1 mole-%).

The ranges for the mole ratios between the selected biorelevant surfactants in (a), (b) and (c) in the biorelevant solid compositions, aqueous concentrates and resulting fasted state biorelevant media are:
The mole ratio of bile salts to phospholipids and 14 carbon to 22 carbon fatty acids or monovalent salts of fatty acids is 1:2 to 20:1.
The mole ratio of bile salts to phospholipids is 1:1 to 20:1, 4:1 to 15:1, or between 8:1 and 15:1.
The mole ratio of PC to 14 carbon to 22 carbon fatty acids or monovalent salts of fatty acid is 1:20 to 20:1, about 1:5 to 5:1, or 1:2 to 2:1.
The mole ratio of monoacyl PC to 14 carbon to 22 carbon fatty acids or monovalent salts of fatty acids is 1:20 to 20:1, about 1:5 to 5:1, or 1:2 to 2:1.
The mole ratio of phospholipids (PC and monoacyl PC) to 14 carbon to 22 carbon fatty acids or monovalent salts of fatty acids (c) is 1:20 to 20:1, about 1:5 to 5:1, or 1:2 to 2:1.
The mole ratio of PC to monoacyl PC is 1:20 to 20:1, about 1:20 to 1:1, or 1:20 to 1:2.

Human Biorelevant Media (FaSSGF-V3 Human and FaSSIF-V3 Human)

Optimized biorelevant media for example comprise or consist of a selection of surfactants targeting surface tension, occurring in the gastrointestinal tract of mammals selected from,
(i) at least one or two bile salts that may be selected from the group consisting of sodium cholate, sodium taurocholate, sodium glycocholate, sodium deoxycholate, sodium taurodeoxycholate, sodium glycodeoxycholate, sodium ursodeoxycholate, sodium chenodeoxycholate, sodium taurochenodeoxycholate, sodium glyco chenodeoxycholate, sodium cholylsarcosinate, sodium N-methyl taurocholate and their free acids,
(ii) at least one phospholipid selected from substantially pure diacyl phospholipids comprising between 60% and 99% by weight phosphatidylcholine (PC); partially enzyme digested diacyl phospholipids containing between 50% and 90% by weight monoacyl PC obtained by back-blending process; mixtures thereof comprising between 5% and 80% by weight monoacyl PC,
(iii) at least one fatty acid or monovalent salt of fatty acid, such as at least one 14 carbon to 22 carbon fatty acid or monovalent salt of 14 carbon to 22 carbon fatty acid.

Optimized biorelevant media targeting surface tension simulating fasted state conditions in human and mammalian species comprise:
(a) at least one bile salt: between 40 and 95 mole-% (at least 40 mole-% and between 40 mole-% and 50 mole-%), (at least 50 mole-% and between 50 mole-% and 60 mole-%), (at least 60 mole-% and between 60 mole-% and 70 mole-%), (at least 70 mole-% and between 70 mole-% and 80-mole %), (at least 80-mole % and between 80 mole-% and 90 mole-%) or (at least 90 mole-% and between 90 mole-% and 95 moles-%).
(b) at least one phospholipid: between 0.1 mole-% and 40 mole-% (between 0.5 mole-% and 30 mole-%, between 1 mole-% and 20 mole-%, between 2 mole-% and 20 mole-%; or between 1 mole-% and 15 mole-%.
Phospholipids comprise:
substantially pure diacyl phospholipids comprising between 60% and 99% by weight PC,
or
(ii) partially enzyme digested diacyl phospholipids containing between 50% and 90% by weight monoacyl PC obtained by back-blending with PC
Or
(iii) mixtures of PC (i) and partially enzyme digested diacyl phospholipids (ii) wherein the level of monoacyl PC is between 5% and 80% by weight.
(c) at least one (such as 14 carbon to 22 carbon) fatty acid or monovalent salt of fatty acid between 0.1 mole-% and 40 mole-% (between 0.5 mole-% and 30 mole-%, between 1 mole-% and 20 mole-%, between 2 mole-% and 20 mole-%; or between 1 mole-% and 15 mole-%,); optionally
(d) cholesterol: between 0 mole-% and 10 mole-%, (between 0.001 mole-% and 10 mole-%, between 0.01 mole-% and 7.5 mole-%, between 0.01 mole-% and 5 mole-%, or between 0.01 mole-% and 1 mole-%).

Targeted surface tension according to the present invention for human and canine FaSSGF and FaSSIF (e.g. FaSSGF-V3 human and FaSSIF-V3 human and FaSSGF-canine and FaSSIF-canine) are consistently between 25 mN/m and 50 mN/m and or in the range between 30 mN/m and 45 mN/m or 30 mN/m and 42 mN/m.

The media composition and concomitant surface tension values are optimized to match as closely as possible the fluids in the stomach and in the target location of the upper small intestine of the given mammal taking into consideration the target surface tension parameter (within the range of 25-50 mN/m).

The prior art has neither considered targeting surface tension to optimize biorelevant media and thus avoid batch to batch variations (due to unknown impurities in the components), nor explicitly point at desired components and the amounts for consistently targeting surface tension between 25 mN/m and 50 mN/m in fasted state media. On the contrary the prior art teaches away from the invention and suggest different grades of the surfactants that can be used in biorelevant media. Further, given that an object of the invention is to provide reproducible media and avoid batch to batch variations, it seems reasonable to select and define components that track the surface tension of gastric and intestinal fluids in humans and canine when optimizing biorelevant media simulating fasted state conditions.

The prior art does not disclose homogeneous solid or concentrated aqueous compositions for providing human FaSSGF and human FaSSIF, canine FaSSGF and canine FaSSIF characterised by unique combinations of analytically defined components targeting surface tension in the range between 25 mN/m and 50 mN/m, between 28 mN/m and 45 mN/m, between 30 mN/m and 45 mN/m, or between 30 mN/m and 42 mN/m.

Biorelevant media which consist of binary mixtures of only bile salts and only diacyl phospholipids form mixed micelles in aqueous medium may not provide solutions or dispersions with surface tension consistently within a range. The fasted state biorelevant media (for example FaSSGF-V3 human and FaSSIF-V3 human) in this invention are defined such that the combination of the selected surfactants expressed in mmol targets surface tension between 25 mN/m and 50 mN/m. It is appreciated that the surface tension in prior art biorelevant media simulating fed state intestinal conditions for example FeSSIF may have surface tension within the range claimed herein. However, prior art fed state media essentially contain lipolysis products fatty acids as well as monoglycerides which are not combined together in the fasted state biorelevant media, such as human FaSSGF and FaSSIF, of present invention (e.g. FaSSGF-V3 human and FaSSIF-V3 human) comprising analytically defined bile salt, phospholipid and fatty acid and consistently targeting surface tension between 25 mN/m and 50 mN/m are not disclosed in prior art fasted state media.

Prior art FaSSIF-Original and FaSSIF-V2 comprise bile salts combined with diacyl phospholipids and does not teach for example, the selection of surfactants in particular monoacyl PC, i.e. lyso PC provided in the form of partially enzyme digested diacyl phospholipids comprising 50% to 90% by weight of monoacyl phospholipids or monoacyl PC and/or fatty acids disclosed herein. Further, the surface tension in the comparative examples shown for prior art FaSSIF-Original is about 52 mN/m (see comparative example 10) and for FaSSIF-V2 about 54 mN/m (see comparative example 9), which are outside the range for human FaSSGF and FaSSIF (FaSSGF-V3 human and FaSSIF-V3 human) of present invention, in particular outside the surface tension range from 25 mN/m to 50 mN/m or the range from 30 mN/m to 45 mN/m or 30 mN/m to 42 mN/m.

According to the present invention biorelevant media (such as e.g. FaSSGF-V3 human and FaSSIF-V3 human) are prepared either for example, by dissolving or dispersing separately weighed amounts of surfactants and optional co-surfactants from scratch in aqueous medium; alternatively, dissolving or dispersing defined amounts of the solid precursor composition, for example a powder, or diluting the liquid precursor composition, for example an aqueous concentrates, in the aqueous medium. Aqueous medium comprise components selected from but not limited to buffers, osmotic components, stabilizers, antioxidants, pH adjusters, antimicrobials, enzymes.

A method of preparation of biorelevant media from scratch (as mentioned herein) involves the steps of (a) mixing water and buffer and optionally other water soluble ingredients, and (b) adding individually weighed surfactants and co-surfactants, such as bile salt, phospholipids and fatty acids, separately one after another or at the same time to aqueous medium resulting from step (a).

Preparing the biorelevant media from solid compositions for example powders, or liquid compositions, for example aqueous concentrates is more cost effective and has the advantage that the media, which have limited stability once prepared in the final form for use, need not be stored. They can be freshly made up instantly in situ as required in desired aqueous medium, with minimum inter-batch variation and weighing inaccuracies. By contrast, making up the media from scratch each time using individually weighed components is not the most cost and time efficient. Furthermore, separating the buffers and osmotic components from the homogeneous solid or liquid compositions confer greater flexibility for selecting and tailoring the media to the desired pH and osmotic pressure in the different locations in the GI tract. The biorelevant compositions (“instant” versions) of the biorelevant media are constituted with surfactants and optional co-surfactants to afford the possibility of combining them with buffers and osmotic agents appropriate to the species and the segment of the gastrointestinal tract to be simulated, as well as variations in physiological conditions at these locations, into consideration. As an illustration, it may be desirable to know how a drug product will perform in humans that are being treated with gastric acid blockers (e.g. proton pump inhibitors) vis a vis in humans with normal, low gastric pH. In such a case, the choice of diluent or aqueous medium will be different for the two situations, although the same biorelevant composition can be used as the starting point for the reconstitution of the biorelevant media. Similarly, in some pre-clinical studies, dogs are administered for example by iv injection, pentagastrin to stimulate gastric acid production while in other studies this is not done and the dogs will have a higher gastric pH. To forecast the in vivo outcome, buffers with appropriate pH, buffer strength and osmolarity can be used to reconstitute the biorelevant composition or to make up the standardized biorelevant media from scratch.

Micelles and mixed micelles comprising mixtures of bile salts and diacyl phospholipids only can have variable surface tension which can be less than 25 mN/m or above 50 mN/m depending on the selection of the molar concentrations and mole ratios of the surfactants in the mixture. The surface tension of water alone is 72.8 mN/m measured at room temperature. Buffers do not significantly affect the surface tension of water. The observation that the surface tension of upper gastrointestinal fluids lies within a band suggest that for consistently simulating surface tension of physiological fluids in the fasted state, biorelevant surfactants other than just bile salts and diacyl phospholipids and, in particular, their mole concentration should be taken into account. This invention describes biorelevant compositions comprising analytically defined selections of components, a method for preparing biorelevant media (in particular FaSSGF and FaSSIF, e.g. FaSSGF-V3 human and FaSSIF-V3 human) simulating fasted state conditions and targeting surface tension consistently between 25 mN/m and 50 mN/m with the object of optimizing biorelevant media simulating fasted state conditions and providing reproducibility. FaSSGF and FaSSIF designed for humans and canine in this invention comprise unique combinations of at least one of each analytically defined bile salt, phospholipid, fatty acid that are neither anticipated nor found in prior art fasted state biorelevant medium.

Without being bound by the explanation, surface tension may result from the interplay between the surfactants in the mixture resulting in colloidal aggregates in the bulk liquid media, for example in the form of micelles, mixed micelles and vesicles. Further, the surfactant mixture may result in some surface active species not being included in colloidal aggregates but existing as monomers below the critical micelle concentration (CMC). This is particularly relevant if crude bile salts and phospholipids are used because of the presence of impurities. Surface tension is exerted at the air/liquid interface or liquid/solid interface and may express the overall aggregation state of the surfactant mixtures depending also on the presence (if any) of impurities. Thus surface tension may be defined by the surfactant mixtures and may be a useful physicochemical parameter to target, both for optimising and checking reproducibility in FaSSGF and FaSSIF (e.g. FaSSGF-V3 human and FaSSIF-V3 human). Further, surface tension is a desirable property because lowering the surface tension leads to an increase in contact (“wetting”) between the fasted state biorelevant media and the surface of poorly soluble drug particles or drug products thereby facilitating dissolution. The prior art has not considered this feature in designing fasted state biorelevant media and optimizing in terms of surface tension consistently between 25 mN/m and 50 mN/m for simulation of fasted state conditions and reproducibility of the media.

Biorelevant Media Simulating Fasted State Conditions in Canine and Other Species

Pre-clinical studies of oral dosage forms are generally carried out in dogs. Other pre-clinical animal species include but are not limited to mouse, rat, rabbit, guinea pig, monkey and pig. Biorelevant media employed presently in early drug development studies in canine models for in vitro-in vivo correlation and prediction, for example FaSSGF-Original, FaSSIF-Original, FaSSIF-V2, had actually been designed for human studies. There are differences in the composition of gastric and intestinal fluids in humans and canine species for example in pH and composition of bile salts and phospholipids in the fasted state. Therefore, it makes sense to provide separate canine biorelevant media for in vitro tests of active pharmaceutical ingredient (API) and formulation performance. It is to be understood that this invention describes in particular human and canine FaSSGF and FaSSIF (e.g. FaSSGF-V3 human and FaSSIF-V3 human, FaSSGF-canine and FaSSIF-canine) simulating fasted state gastric and intestinal fluids across different mammalian species defined by surfactant composition and surface tension within the range of 25 mN/m to 50 mN/m for the specific purpose of in vitro solubility, dissolution and permeability assessments and correlations with in vivo data in a given mammal.

Disclosed for the first time are canine biorelevant media characterised by unique combinations of analytically defined components and physicochemical parameters, particularly surface tension that simulate canine fasted state gastric conditions (herein identified as FaSSGF-canine) and canine fasted state simulated intestinal fluid (herein identified as FaSSIF-canine). Dissolution and solubility data obtained in canine biorelevant media provide better correlation to canine in vivo pharmacokinetic (PK) data compared to using human biorelevant media.

In vitro testing in canine biorelevant media and establishing IVIV correlation in canine can facilitate approval of veterinary products and for comparing in vitro release data in canine media with in vitro data using human media. It can also facilitate rational selection of the most appropriate pre-clinical test species without involving large number of in vivo trials in different species.

According to the prior art, the solubility of the poorly soluble base ketoconazole in prior art FaSSIF-Original (Söderlind) is 26 μg/ml. In dog intestinal aspirates (Kalantzi) the solubility is between 30 and 160 μg/ml. In comparison the solubility in canine FaSSIF (e.g. FaSSIF-canine) is 84.2 μg/ml and therefore within the range found in actual dog aspirates.

The solubility of the poorly soluble base dipyridamole in prior FaSSIF-Original (Söderlind) is 19 μg/ml. In canine intestinal aspirates (Kalantzi) the solubility is between 25 and 95 μg/ml. In comparison, the solubility in canine FaSSIF (e.g. FaSSIF-canine) is 75.0 μg/ml and within the range found in actual dog aspirates.

Cholesterol may be included in the surfactant mixture in amounts up to 10 mole %, for example between 0.001 mole % and 10 mole %. Including cholesterol in human FaSSGF or in human FaSSIF for simulating physiological fluids may provide closer simulation for testing solubility or dissolution of lipophilic drugs and formulations. Whether or not cholesterol is included in biorelevant media to simulate physiological fluids in the fasted state is optional and depends on the drug to be assessed. Co-surfactants for example cholesterol and its esters and amounts between 0% and 10 mole %, or for example between 0.001% and 10 mole % may be included in human FaSSGF (e.g. FaSSGF-V3 human) and human FaSSIF (e.g. FaSSIF-V3 human) and canine FaSSGF (e.g. FaSSGF-canine) and canine FaSSIF (e.g. FaSSIF-canine) media as long as the surface tension is between 25 mN/m and 50 mN/m.

Method for Preparing Human FaSSGF, Human FaSSIF, Canine FaSSGF and Canine FaSSIF (i.e. FaSSGF-V3 Human and FaSSIF-V3 Human, FaSSGF-Canine and FaSSIF-Canine)

Fasted state biorelevant media are obtained from the solid biorelevant compositions for example powder by adding the powder to aqueous medium comprising components selected from water, buffer, pH adjusters, osmotic components, stabilizers, antioxidants, antimicrobials, enzymes for example pepsin or pancreatic enzymes.

Fasted state biorelevant media are obtained by diluting the liquid biorelevant composition for example aqueous concentrates with aqueous medium comprising components selected from water, buffer, pH adjusters, osmotic components, stabilizers, antioxidants, antimicrobials, enzymes for example pepsin or pancreatic enzymes.

FaSSGF-V3 human comprises between 0.01 mmol and 5 mmol, or between 0.01 mmol and 1 mmol of surfactants and optional co-surfactants.

FaSSIF-V3 human comprises between 2 mmol and 20 mmol, 2 mmol to 6 mmol, or 3 mmol to 5 mmol of surfactants and optional co-surfactants.

FaSSGF-canine comprises between 0.01 mmol and 5 mmol, between 0.1 mmol and 1 mmol; or FaSSIF-canine comprising between 2 mmol and 20 mmol (or 10 mmol to 15 mmol, or 12 mmol to 14 mmol) of surfactants and optional co-surfactants.

FaSSIF-canine comprises 5 mmol to 20 mmol, or 10 mmol to 15 mmol of surfactants and optional co-surfactants.

The lower surfactant concentrations for preparing FaSSGF-V3 human and FaSSGF-canine compared to FaSSIF-V3 human and FaSSIF-canine reflect the small amounts found in the stomach due to reflux of intestinal contents.

FaSSIF-V3 human or FaSSIF-canine may optionally comprise between 0.001 mole % and 10 mole % co-surfactant, for example cholesterol.

pH of Human FaSSGF and Human FaSSIF (i.e. FaSSGF-V3 Human and FaSSIF-V3 Human)

The pH of FaSSGF-V3 human is between pH 1 and 3, for example about pH 1.6.

The pH of FaSSIF-V3 human is between 5 and 8, for example about pH 6.8.

pH of Canine FaSSGF and Canine FaSSIF (i.e. FaSSGF-Canine and FaSSIF-Canine)

Furthermore in separate embodiments FaSSGF-canine at pH 1-3, for example pH 1.5 and FaSSGF-canine at pH 5-8, for example pH 6.5 simulate in vitro the physiological gastric fluids of dog which are treated with and without pentagastrin respectively.

FaSSGF-canine is at for example pH 1.5 to test the solubility and dissolution of poorly soluble drugs, particularly acidic drugs, or precipitation of soluble salt forms in stomach juices at acid pH 1.5 or for example at pH 6.5 to test the solubility and dissolution of poorly soluble drugs particularly acid drugs, or precipitation of soluble salt forms in stomach juices at acid pH 6.5 to mimic effects of, for example antacids, H₂ antagonists and inhibitors which suppress acid production in the stomach.

The pH of FaSSIF-canine is between pH 6 to 9, for example pH 7.5.

Optionally 0.1 mg/mL to 1 mg/mL of pepsin may be added to FaSSGF-V3 human or FaSSGF-canine.

Osmolarity and Buffer Capacity Human FaSSIF (i.e. FaSSIF-V3 Human)

The osmolarity of FaSSIF-V3 human is in the range between 175 mOsm/kg and 280 mOsm/kg, or between 130 mOsm/kg and 225 mOsm/kg—for example about 200 mOsm/kg.

The buffer capacity of FaSSIF-V3 human is in the range between 2.5 mmol/l/ΔpH and 6.0 mmol/l/ΔpH, or between 3 mmol/l/ΔpH and 5.8 mmol/l/ΔpH for example about 5.6 mmol/l/ΔpH.

Osmolarity and Buffer Capacity Canine FaSSIF (FaSSIF-Canine)

The osmolarity of FaSSIF-canine is in the range between 25 mOsm/kg and 600 mOsm/kg, between 50 mOsm/kg and 300 mOsm/kg, or between 100 mOsm/kg and 250 mOsm/kg, for example 180 mOsm/kg.

The buffer capacity of FaSSIF-canine is in the range between 1.0 mmol/l/ΔpH and 50 mmol/l/ΔpH, between 2 mmol/l/ΔpH and 30 mmol/l/ΔpH, or between 5 mOsm/kg and 15 mOsm/kg for example about 10 mmol/l/ΔpH.

Osmolarity and Buffer Capacity Human FaSSGF (i.e. FaSSGF-V3 Human)

The osmolarity of FaSSGF-V3 human is in the range between 10 mOsm/kg and 400 mOsm/kg, between 25 mOsm/kg and 300 mOsm/kg, or between 50 mOsm/kg and 200 mOsm/kg, for example about 120 mOsm/kg.

The buffer capacity of FaSSGF-V3 human is in the range between 0 mmol/l/ΔpH and 50 mmol/l/ΔpH, between 0 mmol/l/ΔpH and 30 mmol/l/ΔpH, between 0 mOsm/kg and 10 mOsm/kg.

Osmolarity and Buffer Capacity Canine FaSSGF (i.e. FaSSGF-Canine)

The osmolarity of FaSSGF-canine is in the range between 10 mOsm/kg and 400 mOsm/kg, preferably between 25 mOsm/kg and 200 mOsm/kg, or between 50 mOsm/kg and 150 mOsm/kg, for example about 100 mOsm/kg.

The buffer capacity of FaSSGF-canine is in the range between 1 mmol/l/ΔpH and 50 mmol/l/ΔpH, between 2 mmol/l/ΔpH and 30 mmol/l/ΔpH, or between 5 mOsm/kg and 15 mOsm/kg for example about 10 mmol/l/ΔpH.

Method for Preparing Solid Compositions and Aqueous Concentrates

The method for preparing solid biorelevant compositions includes a step which comprises dissolving the surfactants and optionally co-surfactants in a solvent, water or mixtures thereof and eliminating the solvent, thereby providing a homogeneous solid composition wherein the moisture content is below 5% by weight, or below 3% by weight.

The dried solid composition is milled and screened or sieved to obtain a powder composition with mean particle diameter between 10 μm and 1000 μm or 50 μm to 500 μm; bulk density between 0.3 g/m³ and 0.7 g/cm; moisture content below 5% by weight; granules; pellets with mean particle diameter 200 to 2000 μm; tablets; or capsules.

Alternatively, the method for preparing homogeneous aqueous concentrate comprising between 5% and 60%, 10% to 40% or 10% to 30% by weight of the surfactants and optional co-surfactants includes a step which consists of homogeneously dissolving or dispersing the surfactants and optionally co-surfactants in water at a temperature between 15° C. and 60° C. without a drying step to remove the water.

Fasted state biorelevant media are also directly obtained by individually weighing and dissolving the surfactants and optionally co-surfactants separately, together or sequentially in the aqueous media comprising components selected from water, buffer, osmotic components, stabilizers, antioxidants, pH adjusters, antimicrobials.

Typical analytically defined components for making fasted state biorelevant media are detailed below.

Bile Salts are selected from sodium cholate, sodium taurocholate, sodium glycocholate, sodium deoxycholate, sodium taurodeoxycholate, sodium glycodeoxycholate, sodium ursodeoxycholate, sodium chenodeoxycholate, sodium taurochenodeoxycholate, sodium glyco chenodeoxycholate, sodium cholylsarcosinate, sodium N-methyl taurocholate and their free acids. The cholates may be from natural, synthetic or semi-synthetic sources. If the cholate is natural, it should be from porcine or TSE/BSE-free bovine sources typically containing a minimum of about 95% cholate.

Phospholipids are obtained from for example, egg yolk; soy bean; milk; sunflower; oat comprising phosphatidylcholine (PC), phosphatidylethanolamine (PE); phosphatidylserine (PS); phosphatidic acid (PA), phosphatidylinositiol (PI); phosphatidylglycerol (PG). Phospholipids include diacyl and monacyl phospholipid.

Diacyl phospholipids specified in the specification comprise between 60%, <80% or between 90% and 99% by weight phosphatidylcholine (PC) with fatty acid chains having between 14 and 24 carbon atoms.

Partially enzyme digested diacyl phospholipids comprise monoacyl PC (between 50% and 90% by weight), PC and less than 5% by weight concomitant components obtained by a back blending process with PC. Partially enzyme digested diacyl phospholipids is used for providing monoacyl phospholipids and in particular monoacyl PC in this invention.

It is difficult to control fixed levels of monoacyl PC directly by enzyme digestion. Back-blending is a method for obtaining analytically defined amounts of monoacyl PC by titration, using 98% to 99% by weight (pure) PC and a solution of partially enzyme digested diacyl phospholipids (such as after purification) comprising more than the amount of monoacyl PC necessary in the end product. After eliminating the solvent, the homogeneous solid mixture comprises a defined amount of monoacyl PC.

Fatty acids are selected from the group comprising at least one 14 carbon to 22 carbon fatty acid or monovalent salt of fatty acid for example myristic acid, palmitic acid, stearic acid, oleic acid, arachidic acid, behenic acid.

Monovalent salts of fatty acid comprise the sodium or potassium salts of fatty acids from the list above comprising at least 97% of the dried sodium or potassium salt form. Typically, sodium oleate comprises at least 85% oleic acid.

Cholesterol: cholesterol and cholesterol esters, comprising at least 80% by weight, 90% by weight, or at least 95% by weight of cholesterol or cholesterol ester.

Molecular weight used for calculating molar concentrations and molar ratios.

Sodium taurocholate 538

Monoacyl phospholipid 505

Diacyl phospholipid 787

Sodium oleate 304

Oleic acid 282

Cholesterol 387

Buffers and pH: Exemplary buffer media to maintain pH at 1.5; 6.5 and 7.5 are described but not limited to Examples 11 to 13

Osmotic components: Exemplary osmotic components comprise but not limited to sodium chloride

Method of Preparing Solid Biorelevant Compositions

The desired amount of bile salts, phospholipid, fatty acid or monovalent salts, optionally cholesterol are dissolved in a solvent or water, and mixtures of solvent. Selected solvents are methanol, ethanol, tertiary butanol and combinations of hydrophilic solvents or dichloromethane on its own. Solutions of tertiary butanol and water in equal amounts are particularly desired.

After the surfactants are completely dissolved the clear, white to yellowish solution is freeze-dried using a Christ Epsilon 2-4 LSC lyophilizer.

Alternatively the solution is spray dried.

The moisture content of the lyophilised solid is below 5% by weight.

The solid is converted to a particulate composition by milling or grinding to a mean particle size range between 10 μm and 1000 μm.

After milling and screening the powder is ready to use for preparing fasted state biorelevant media by dissolving the desired molar concentration in the buffer solutions comprising osmotic components as shown in the examples.

Physical Characteristics of the Powders

The mean particle size is in the range between 10 μm to 1000 μm.

The bulk density is between 0.3 g/cm³ to 0.7 g/cm³.

The moisture content is below 5% by weight or less than 3% by weight.

Method of Preparing Liquid Biorelevant Composition (Aqueous Concentrate)

The desired amount by weight of bile salts, phosholipid, fatty acid or monovalent salts, comprising between 5% and 60%, 10% to 40%, or 10% to 30%, by weight of the surfactants, optionally cholesterol, are dissolved in water at a temperature between 15° C. and 60° C. Stabilizers for example sodium azide, thiomersal, EDTA, tocopherols may be included in the aqueous solution. After cooling to room temperature and filtration using for example a 0.22 μm filter, the aqueous concentrate may be used to prepare fasted state biorelevant media using the desired molar concentration in the buffer solutions comprising osmotic components.

Physical Characteristics of the Aqueous Concentrate

Z average particle size measured using PCS after diluting 1% by weight of the liquid biorelevant composition to biorelevant medium: range 2 nm to 1000 nm.

Optical properties: Visually clear

Method for Measuring Surface Tension:

Surface tension measurements are carried out in a Kibron AquaPi tensiometer based on the DuNouy principle.

The instrument is pre-calibrated for a temperature of 20° C. A correction factor is applied to any deviation.

The titanium rod is flamed to vaporize impurities before surface tension measurements are taken.

The sample cups are cleaned with ethanol and with purified water.

Recalibrations are carried out every time that the probe is changed or at least daily.

Measurements are done in duplicate for calculating the average and the standard deviation. If the sample temperature deviates from 20° C. the temperature correction factor is taken into consideration.

### Example 1

### Preparation of a Typical Biorelevant Powder Composition for Making Human FaSSIF (i.e FaSSIF-V3 Human)

About 2 g of the solid biorelevant powder composition for the preparation of FaSSIF-V3 human is prepared by dissolving 1.622 g of sodium taurocholate in 10 ml of purified water at room temperature using a magnetic stirrer. After the sodium taurocholate is completely dissolved and a clear solution is obtained 10 ml of tert-butanol is added to the solution. In the next step 0.009 g of diacyl phospholipid (in particular PC) and 0.199 g of monoacyl phospholipid (in particular from partially enzyme digested diacylphospholipids comprising between 50% and 90% monoacyl PC by back-blending) is dissolved in the solution (alternatively the lipids are added in separate steps). After the lipid components are completely dissolved and a clear to slightly yellowish solution is obtained, 0.128 g of sodium oleate is added to the solution. The clear to slightly yellowish solution is transferred into a suitable container for freeze-drying.

### Example 2

### Making Human FaSSIF (i.e. FaSSIF-V3 Human) from the Solid Biorelevant Composition Shown in Example 1

| | | | |
|---|---|---|---|
| Sodium taurocholate | 1.4 | mmol | 0.759 g/l |
| Sodium glycocholate | 1.4 | mmol | 0.683 g/l |
| Diacylphospholipids | 0.035 | mmol | 0.007 g/l |
| (in particular PC) | | | |
| Monoacylphospholipids | 0.315 | mmol | 0.186 g/l |
| (in particular *Monoacyl PC) | | | |
| Sodium oleate | 0.35 | mmol | 0.120 g/l |

| | |
|---|---|
| pH | 6.7 (maleate buffer) |

| | | |
|---|---|---|
| Surface tension | 37.7 | mN/m |

*partially enzyme digested diacylphospholipids comprising about 80% by wt of monoacyl PC

1.741 g of the homogeneous powder composition from example 1 is dissolved in the maleate buffer comprising buffer and osmotic agents (example 14). The pH of the biorelevant medium is adjusted to pH 6.7.

Alternatively the equivalent amount by weight of the surfactants in a liquid composition for example an aqueous concentrate comprising 10% to 60% by weight of surfactants and optionally co-surfactants may be used in place of the powder composition.

Alternatively the components may be added separately.

### Example 3

### Making Human FaSSIF (i.e. FaSSIF-V3 Human) from the Solid Biorelevant Composition Shown in Example 1

| | | | |
|---|---|---|---|
| Sodium taurocholate | 2.8 | mmol | 1.518 g/l |
| Diacylphospholipids | 0.035 | mmol | 0.007 g/l |
| (in particular PC) | | | |
| Monoacylphospholipids | 0.315 | mmol | 0.186 g/l |
| (in particular *Monoacyl PC) | | | |
| Sodium oleate | 0.35 | mmol | 0.120 g/l |

| | |
|---|---|
| pH | 6.5 (maleate buffer) |

| | | |
|---|---|---|
| Surface tension | 34.7 | mN/m |

*partially enzyme digested diacylphospholipids comprising about 80% by wt of monoacyl PC

1.87 g of the homogeneous powder composition from example 1 is dissolved in the maleate buffer comprising buffer and osmotic agents (example 13). The pH of the biorelevant medium is adjusted to pH 6.5.

Alternatively, 3.5 mmol of surfactants contained in an aqueous concentrate comprising 10% to 60% by weight of surfactants may be used in place of the powder composition.

Alternatively the components may be added separately to prepare fasted state media.

### Example 4

### Preparation of a Biorelevant Powder Composition for Making Canine FaSSGF and Canine FaSSIF (i.e. FaSSGF-Canine and FaSSIF-Canine)

2.00 g of the solid biorelevant powder composition is prepared by dissolving 0.727 g of sodium taurocholate and 0.711 g of sodium taurodeoxycholate in 10 ml of purified water at room temperature using a magnetic stirrer. Alternatively the bile salts are added in separate steps and completely dissolved until a clear solution is obtained. 10 ml of tert-butanol is added to the solution. In the next step 0.249 g of diacylphospholipids (in particular PC) and 0.198 g of monoacylphospholipids (in particular monoacyl PC from partially enzyme digested diacylphospholipids comprising between 50% and 90% monoacyl PC by back blending) are dissolved in the solution. After the lipid components are completely dissolved and a clear to slightly yellowish solution is obtained, 0.115 g of sodium oleate is added. The clear to slightly yellowish solution is transferred into a suitable container for freeze-drying.

### Example 5

### Making Canine FaSSGF (i.e. FaSSGF-Canine)

0.149 g of a homogeneous powder composition from example 3 (comprising sodium taurocholate, sodium taurodeoxycholate, diacylphospholipids (in particular diacyl PC), monoacylphospholipids (in particular monoacyl PC) and sodium oleate) is dissolved in either a 1 liter of phosphate buffer comprising buffer and osmotic agents (example 11) or 1 liter of a pH 1.5 non-buffered HCl solution comprising an osmotic component (example 15). The pH of the biorelevant medium is adjusted to either pH 6.5 or 1.5.

| | | |
|---|---|---|
| Sodium taurocholate | 0.1 mmol | 0.054 g/l |
| Sodium taurodeoxycholate | 0.1 mmol | 0.053 g/l |
| Diacylphospholipids (in particular PC) | 0.025 mmol | 0.019 g/l |
| Monoacylphospholipids | 0.025 mmol | 0.015 g/l |
| (in particular *Monoacyl PC) | | |
| Fatty acids or monovalent salts | 0.025 mmol | 0.009 g/l |

| | |
|---|---|
| pH | **1.5 (±0.25) (dogs treated |
| | with Pentagastrin) and |
| | ***6.5 (±0.25) (dogs not treated |
| | without Pentagastrin) |
| Surface tension | 35.0 mN/m |

*partially enzyme digested diacylphospholipids comprising about 80% by wt of monoacyl PC

### Example 6

### Making Canine FaSSIF (i.e. FaSSIF-Canine)

7.46 g of a homogeneous powder composition from example 3 (comprising sodium taurocholate, sodium taurodeoxycholate, diacyl phospholipids, monoacyl phospholipids and sodium oleate) is dissolved in 1 liter of phosphate buffer (example 12) comprising buffer and osmotic agents. The pH of the biorelevant medium is adjusted to pH 7.5.

| | |
|---|---|
| Sodium taurocholate | 5.00 mmol |
| Sodium taurodeoxycholate | 5.00 mmol |
| Diacylphospholipids (in particular PC) | 1.25 mmol |
| Monoacylphospholipids (in particular | 1.25 mmol |
| *Monoacyl PC) | |
| Fatty acids or monovalent salts | 1.25 mmol |
| pH: | 7.5 |
| Surface tension | 40.6 mN/m |

*partially enzyme digested diacylphospholipids comprising about 80% by wt of monoacyl PC

### Example 7

### FaSSIF Composition (i.e. FaSSIF-V3 Human) with Diacylphospholipids and Fatty Acids, in Particular with PC and Fatty Acids

| | |
|---|---|
| Sodium taurocholate | 3.00 mmol |
| Diacylphospholipids | 0.75 mmol |
| (in particular PC) | |
| Sodium oleate | 0.75 mmol |
| pH | 6.5 (phosphate buffer) |
| Surface tension | 39.3 mN/m |

### Example 8

### FaSSIF Composition (i.e. FaSSIF) with Monoacyl- and Diacylphospholipids, in Particular with PC and Monoacyl PC

| | |
|---|---|
| Sodium taurocholate | 3 mmol |
| Diacylphospholipids (in particular PC) | 0.075 mmol |
| Monoacylphospholipids | 0.675 mmol |
| (in particular *Monoacyl PC) | |
| pH | 6.5 (phosphate buffer) |
| Surface tension | 42.3 mN/m |

*partially enzyme digested diacylphospholipids comprising about 80% by wt of monoacyl PC

### COMPARATIVE EXAMPLES

### Example 9

### FaSSIF V2 (Jantratid 2008)

| | |
|---|---|
| Sodium taurocholate | 3 mmol |
| Diacyl phospholipids | 0.2 mmol |
| pH | 6.5 (maleate buffer) |
| Surface tension | 54.3 mN/m |

### Example 10

### FaSSIF Original (WO 2007/054342)

| | |
|---|---|
| Sodium taurocholate | 3 mmol |
| Diacylphospholipids | 0.75 mmol |
| pH | 6.5 (phosphate buffer) |
| Surface tension | 53.3 mN/m |

### Example 11

### Blank Phosphate Buffer pH 6.5 for preparing FaSSIF-Original

| | |
|---|---|
| Sodium dihydrogen phosphate | 28.65 mmol |
| Sodium hydroxide | 10.5 mmol |
| Sodium chloride | 105.85 mmol |
| pH | 6.5 |
| Surface Tension | 73.6 mN/m |

### Example 12

### Blank Phosphate Buffer pH 7.5 for Preparing Canine FaSSIF (i.e. FaSSIF-Canine)

| | |
|---|---|
| Sodium dihydrogen phosphate | 28.65 mmol |
| Sodium hydroxide | 21.66 mmol |
| Sodium chloride | 39.14 mmol |
| pH | 7.5 |
| Surface Tension | 72.9 mN/m |

### Example 13

### Blank Maleate Buffer pH 6.5 for preparing a FaSSIF (i.e. FaSSIF-V3 human)

| | |
|---|---|
| Maleic acid | 21.68 mmol |
| Sodium hydroxide | 40.23 mmol |
| Sodium chloride | 23.12 mmol |
| pH | 6.5 |
| Surface Tension | 73.4 mN/m |

### Example 14

### Blank Maleate Buffer pH 6.7 for Preparing a FaSSIF (i.e. FaSSIF-V3 Human

| Maleic acid | 10.27 mmol |
|---|---|
| Sodium hydroxide | 16.55 mmol |
| Sodium chloride | 93.3 mmol |
| pH | 6.7 |

### Example 15

### HCl pH 1.5 for Preparing Canine FaSSGF (i.e. FaSSGF-Canine)

| | |
|---|---|
| Hydrochloric acid | q.s. pH 1.5 |
| Sodium chloride | 20.00 mmol |
| pH | 1.5 |
| Surface Tension | 72.8 mN/m |

Contribution by the Invention

The invention discloses optimized biorelevant media simulating fasted state conditions in the stomach and the upper small intestine of a given mammalian species, including but not limited to human and canine models. Optimized FaSSGF and FaSSIF (such as of the type FaSSGF-V3 human and FaSSIF-V3 human) according to present invention are specific examples of fasted state biorelevant media prepared using defined amounts of the solid composition or liquid concentrate or from scratch, for example by dissolving or dispersing the components separately in aqueous media.

Fasted state biorelevant media according to present invention (such as of the types FaSSGF-V3 human, FaSSIF-V3 human, FaSSGF-canine and FaSSIF-canine) are further defined by analytically specified components and consistently target surface tension broadly between 25 mN/m and 50 mN/m. Such biorelevant media are highly reproducible and valuable tools for in vitro dissolution testing in pre-clinical development, formulation optimization, de-risking bioequivalence bridging studies, and in modelling and simulation.

The compositions are defined by (a) at least one bile salt (b) at least one phospholipid which may be PC; and/or monoacyl PC from enzyme digested diacylphospholipids comprising between 50% and 90% by weight monoacyl PC; Prior art biorelevant media are not optimized in that the disclosed compositions do not contain fatty acids and/or monoacyl PC provided in the form of partially enzyme digested diacyl phospholipids.

The prior art has not positively targeted surface tension in the biorelevant media for in vitro testing. Surface tension is a desirable property because lowering the surface tension leads to an increase in contact (“wetting”) between the biorelevant media and the surface of poorly soluble drug particles or drug products thereby facilitating dissolution.

None of the fasted state biorelevant media disclosed in the prior art contains exactly the same selections or proportions of analytically defined surfactants that are claimed presently (especially of the types FaSSGF-V3 human, FaSSIF-V3 human, FaSSGF-canine and FaSSIF-canine). The prior art does not positively state that surface tension of such media be targeted and optimized to provide reproducible biorelevant medium simulating fasted state gastric and upper intestinal fluids for solubility tests. Particularly, assessing dosage forms for batch to batch variation and confirmation of reproducibility when screening formulations for in vivo evaluation.

It is also advantageous that the media be easily and reproducibly prepared in an efficient manner as this will lead to more reliable results and thereby better forecasting of in vivo drug performance. Moreover, there is an unmet need for optimizing media by selecting analytical quality surfactants to target surface tension and in turn characterised by surface tension parameter between 25 mN/m and 50 mN/m and can be implemented with assurance of reproducibility in laboratories globally.

Biorelevant media currently employed in early drug development studies in canine models for in vitro-in vivo correlation and prediction, for example FaSSGF-Original, FaSSIF-Original, FaSSIF-V2, have actually been designed for human studies and are not optimized in terms of the proposed surface tension parameter in the range of 25 mN/m to 50 mN/m. The present invention provides canine biorelevant media that can be used for veterinary in vitro bridging assessments, thereby minimizing the number of in vivo studies in dogs.

1. A homogeneous biorelevant composition for preparing fasted state biorelevant media having a surface tension between 25 mN/m and 50 mN/m for simulating fasted state gastric and fasted state upper small intestinal fluids of mammalian species, comprising a plurality of surfactants, comprising:
at least one bile salt;
at least one phospholipid selected from the group of phospholipids comprising:
between 60% and 99% by weight phosphatidylcholine (PC),
partially enzyme digested diacyl phospholipids containing between 50% and 90% by weight monoacyl PC, preferably obtained by back-blending with PC, and
mixtures of PC and partially enzyme digested diacyl phospholipids wherein the level of monoacyl PC is between 5% and 80% by weight; and
at least one fatty acid or monovalent salt of the fatty acid.

2. The homogeneous composition of claim 1, wherein 40 mole-% to 95 mole-% of said plurality of surfactants comprise at least one bile salt and a remaining mole-% of said plurality of surfactants comprises the at least one phospholipid and the at least one fatty acid or monovalent salt of the fatty acid.

3. The homogeneous composition of claim 2, wherein the remaining mole-% of the plurality of surfactants further comprises cholesterol.

4. The homogeneous composition of claim 1, wherein the at least one bile salt is selected from the group consisting of sodium cholate, sodium taurocholate, sodium glycocholate, sodium deoxycholate, sodium taurodeoxycholate, sodium glycodeoxycholate, sodium ursodeoxycholate, sodium chenodeoxycholate, sodium taurochenodeoxycholate, sodium glyco chenodeoxycholate, sodium cholylsarcosinate, sodium N-methyl taurocholate and their respective free acids.

5. The homogeneous composition of claim 1, wherein the at least one fatty acid is at least one of 14 carbon to 22 carbon fatty acid.

6. The homogeneous composition of claim wherein biorelevant composition is devoid of monoglyceride.

7. The homogeneous composition of claim 1 wherein the biorelevant composition is in the form of a solid

8. The homogeneous composition of claim 1 wherein the biorelevant composition is in the form of a liquid composition.

9. An aqueous biorelevant media for simulating fasted state gastric and fasted state upper small intestinal fluids of mammalian species, comprising:
a plurality of surfactants occurring in the gastrointestinal tract of mammals, the plurality of surfactants comprising:
at least one bile salt;
at least one phospholipid selected from the group of phospholipids comprising
between 60% and 99% by weight phosphatidylcholine (PC);
partially enzyme digested diacyl phospholipids containing between 50% and 90% by weight monoacyl PC; and
mixtures of PC and partially enzyme digested diacyl phospholipids wherein the level of monoacyl PC is between 5% and 80% by weight; and
(iii) at least one fatty acid or monovalent salt of the fatty acid, and
wherein the biorelevant media has a surface tension between 25 mN/m and 50 mN/m.

10. The aqueous biorelevant media of claim 9, wherein the surface tension is between 35 mN/m and 45 mN/m, between 28 mN/m and 45 mN/m or between 30 mN/m and 42 mN/m.

11. The aqueous biorelevant media of claim 9, wherein 40 mole-% to 95 mole-% of said plurality of surfactants comprises the at least one bile salt, and that a remaining mole-% of said plurality of surfactants comprises the at least one phospholipid and the at least one fatty acid or monovalent salt of the fatty acid.

12. The aqueous biorelevant media of claim 11 wherein the remaining mole-% of the plurality of surfactants further comprises cholesterol.

13. The aqueous biorelevant media of claim 9, wherein the at least one bile salt is selected from the group consisting of sodium cholate, sodium taurocholate, sodium glycocholate, sodium deoxycholate, sodium taurodeoxycholate, sodium glycodeoxycholate, sodium ursodeoxycholate, sodium chenodeoxycholate, sodium taurochenodeoxycholate, sodium glyco chenodeoxycholate, sodium cholylsarcosinate, sodium N-methyl taurocholate and their respective free acids.

14. The aqueous biorelevant media of claim 9, wherein the at least one fatty acid comprises at least one of 14 carbon to 22 carbon fatty acid.

15. The aqueous biorelevant media of claim 9, wherein the biorelevant media is devoid of monoglyceride.

16. The aqueous biorelevant media of claim 9, wherein a total amount of the plurality of surfactants for simulating human FaSSGF is between 0.01 mmol and 5 mmol or between 0.01 and 1 mmol.

17. The aqueous biorelevant media of claim 9, wherein a total amount of the plurality of surfactants for simulating human FaSSIF is between 2 and 20 mmol, or between 2 and 6 mmol.

18. The aqueous biorelevant media of claim 9 wherein a total amount of the plurality of surfactants for simulating canine FaSSGF is between 0.1 and 5 mmol, between 0.1 and 2 mmol, or between 0.01 mmol and 5 mmol.

19. The aqueous biorelevant media of claim 9, wherein the total amount of the plurality of surfactants for simulating canine FaSSIF is between 2 and 20 mmol, between 5 and 20 mmol, or between 10.0 mmol and 15.0 mmol.

20. The biorelevant media of claim 9, comprising at least 60 mole-% or at least 70 mole-% of the at least one bile salt.

21. The aqueous biorelevant media of claim 9, wherein a mole ratio of a mole sum of monoacyl PC and diacyl PC to the at least one fatty acid, including monovalent salts of the at least one fatty acids, is 1:20 to 20:1.

22. The aqueous biorelevant media of claim 9, wherein a mole ratio of diacyl PC to monoacyl PC is 1:20 to 20:1.

23. The aqueous biorelevant media of claim 9, wherein a mole ratio of diacyl PC to the at least one fatty acid, including monovalent salts of the at least one fatty acid, is 1:20 to 20:1.

24. The aqueous biorelevant media of claim 9, further comprising a plurality of components selected from water, buffer, osmotic components, stabilizers, antioxidants, pH adjusters, antimicrobials, enzymes for example pepsin, pancreatic enzymes.

25. A method of reconstituting a biorelevant media, comprising:
adding defined amounts of a biorelevant composition to water or aqueous media, the biorelevant composition having a surface tension between 25 mN/m and 50 mN/m for simulating fasted state gastric and fasted state upper small intestinal fluids of mammalian species and comprising:
a plurality of surfactants, comprising:
at least one bile salt;
at least one phospholipid selected from the group of phospholipids comprising:
between 60% and 99% by weight phosphatidylcholine (PC),
partially enzyme digested diacyl phospholipids containing between 50 and 90% by weight monoacyl PC, preferably obtained by back-blending with PC, and
mixtures of PC and partially enzyme digested diacyl phospholipids wherein the level of monoacyl PC is between 5% and 80% by weight; and
at least one fatty acid or monovalent salt of the at least one fatty acid.

26. The method of claim 25, further comprising preparing the biorelevant composition as a solid in the form of a powder, granule or capsule by dissolving the plurality of surfactants in a solvent, water or mixtures thereof and eliminating the solvent, water or mixtures thereof thereby producing a solid composition wherein the moisture content of the solid composition is below 5% by weight.

27. The method of claim 25, wherein between 10% and 60% by weight of the plurality of surfactants are homogeneously dissolved or dispersed in aqueous medium comprising further components selected from buffer, osmotic components, stabilizers, antioxidants, pH adjusters, and antimicrobials at a temperature between 15° C. and 60° C. without a drying step to remove the water.

28. The method of claim 25, further comprising a step of adding aqueous medium to the biorelevant composition the aqueous medium comprising a plurality of buffers and osmotic regulators.

29. The method of claim 25, comprising individually weighing and dissolving the plurality of surfactants and any additional co-surfactants separately, together or sequentially in aqueous medium comprising components selected from water, buffer, osmotic components, stabilizers, antioxidants, pH adjusters, and antimicrobials and enzymes for example pepsin, pancreatic enzymes.

30. The method of claim 25, further comprising adding specified proportions of analytically defined surfactants for solubility testing, dissolution testing, bioequivalence assessments, drug release assessments, IVIVC, in silico modelling and simulation, drug supersaturation, drug precipitation, drug stability, performance of enhanced formulations and drug permeability studies.
